(19) United States
(12) Patent Application Publication
(10) Pub. No.: US 2022/0000907 A1
(43) Pub. Date: Jan. 6, 2022

(54) MOUTHWASH/GARGLE, NASAL/ORAL/NASAL SPRAY COVID AND BACTERIOCIDE ARRANGEMENT

(71) Applicants: Eugene Arthur Rosov, South Miami, FL (US); Lukas J. Rosov, Portland, ME (US)

(72) Inventors: Eugene Arthur Rosov, South Miami, FL (US); Lukas J. Rosov, Portland, ME (US)

(21) Appl. No.: 17/189,455

(22) Filed: Mar. 2, 2021

Related U.S. Application Data
(60) Provisional application No. 63/048,092, filed on Jul. 4, 2020.

(51) Int. Cl. A61K 31/79; A61K 9/00; A61K 47/20; A61K 47/10; A61K 9/107

(52) U.S. Cl. CPC A61K 31/79; A61K 9/0056; A61K 9/1075; A61K 9/0043; A61K 47/10; A61K 47/20

(57) ABSTRACT

An ethanol-free antimicrobial treatment composition, comprising: an antimicrobially effective amount of polyvinylpyrrolidone iodine (PVPI) ranging from 1.0% to a dilution-accommodating 3.5%; an inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes; a Stevia sweetener pre-mixed in a microemulsion solution of dimethyl sulfoxide (DMSO); and

ion-free distilled water.

The invention provides a unique healthcare product pursuant to an ethanol-free oral/nasal/ocular care program and includes the option for a vaporized inhalant compositional additive comprising appropriately diluted (commercially available 10% antiseptic polyvinyl pyrrolidone iodine diluted to approximately 1%-1.25%, with approximately 0.1% - 0.125% available iodine) polyvinyl pyrrolidone iodine solution which does not have an unacceptably bitter taste, e.g. as an additive and core component of mouthwashes, lozenges, toothpastes, throat sprays, and breath sprays in any form whatsoever: liquid, gel, cream, aerosol, paste, that are free of ethanol, e.g., comprising:
(i) an antimicrobially effective amount and dilution of liquid polyvinyl pyrrolidone iodine (PVPI) (it is important to add that dozens of researchers and the manufacturers' research indicates that a 1.0% solution of PVPI—with 0.1% available iodine—is by far the most reliably effective. Efficacy in microbial killing for PVPI follows a bell curve and the peak of that curve is directly at a 1.0% concentration);
(ii) one or more flavoring oils which are substantially insoluble in water at room temperature, but which are readily dissolved in
(iii) DMSO—dimethyl sulfoxide, a key diluent, vasodilator, and penetrating/solvent component of the invention mixed with the sweeteners and essential vasodilation flavoring oils prior to incorporation into PVPI and,
(iv) water, wherein the ratio of the PVPI to the one or more flavoring and vasodilation oils (such as peppermint, menthol, eucalyptus and any other “essential” oil is generally from 1:0.005 to 1.0:0.05,
(v) powdered stevia sweetener or other sweetener choices, including alcohol sugars such as xylitol, erythritol, etc.; as well as methods of mixing, making and deploying the same.

### FIELD OF THE INVENTION

The present invention is based upon Provisional Application No. 63/048,092, filed on 5 Jul. 2020, incorporated herein by reference, which invention relates to a novel liquid alcohol-free oral/nasal, ocular, and naso-pharyngeal care composition comprising polyvinyl pyrrolidone iodine (PVPI) as its key active antimicrobial ingredient, combined with water, DMSO, essential/flavorful oils (e.g., peppermint oils) and a non-nutritive sweetener (e.g., Stevia). The delivery methods for the invention are part-and-parcel of the invention's novelty. These include methods for spraying and misting the product into the nose, throat and mouth; eyedrops for drops into the eyes; gargling the product as with a mouthwash and spitting out; a special fan-type sprayer to provide a fine aerosol mist for PPE equipment, with a 170-180-degree aerosol spray to rapidly spray masks and other PPE devices; and infused into a lozenge for irrigating the mouth and throat.

Pursuant to the need for amplifying the capture and killing ability of ordinary personal protective masks, the present invention also incorporate an ampule-like delivery system in which a small (3 cm in diameter; 6 cm in length) vertically or horizontally placed squeezable ampule, fashioned with tiny pinhole apertures on one side, can be squeezed one or more times, and deliver a fine misting spray to the surface of the exterior of the mask, which spray will dry immediately and yet produce an efficient, effective and long-lasting barrier that will kill any microbes which land upon it.

A key feature of the liquid product is that its antimicrobial operative killing longevity, unlike alcohol, has been determined to be over 3 hours, and may extend to as many as 8 hours of killing capability. In over 80 years of use, unlike alcohol, PVPI in various forms and mixtures has never been perceived to demonstrate or encourage antimicrobial resistance, a central concern for domestic and international institutions involved in global and regional healthcare.

### BACKGROUND OF THE INVENTION

This invention applies to the prevention, disablement and in some cases the cure of a broad range of respiratory-related diseases often initially of nasopharyngeal origin caused and exacerbated by infectious and pathogenic microbial organisms.

While many of the most highly transmissible and pandemically dangerous of these microbes are often viruses, such infectious disease organisms include many different types and many different serovars/mutations/variants of bacteria, fungi, molds, archaea and yeasts. These various and always numerous serovars, mutations and variants have arisen for two principal reasons: first, that it is in the nature of rapidly reproducing populations of microbes to produce many thousands of mutations, a few of which are viable, and some of which are more transmissible and of greater morbidity and mortality than others; and second, that the human use and over-use, misuse and negligent deployment of vaccines, antibiotics, and other agents to combat infectious disease organisms, especially in the realm of animal husbandry and the vast global activities involved in global industrial farming, have oftentimes effectuated the development of antimicrobial resistant (AMR) strains of microbes which are unaffected by our most powerful and previously successful antibiotics, and these organisms, in their multiplicitous mutations, can sometimes jump zoonotically from animals to human, and sometimes through multiple zoonotic channels. The direct relationship between industrial farming and the arising of pandemic-potential micro-organisms is well-established in the research literature. At the time of this writing, 40% of the arable land on the earth is devoted to industrial farming.

Almost all known antibiotics and almost all known antibiotic chemicals (e.g., ethanol and isopropanol), occasion the development of AMR (antimicrobial resistant) strains of microbes, which can readily become the dominant, and therefore the most dangerous strains in terms of causing morbidity and mortality—of both animals and man. Despite their ubiquitous use, and indeed overuse of alcohol products such as ethanol, methanol and isopropanol in various healthcare settings, alcohols have been found recently to perpetrate the development of alcohol-resistant microbes, another in a string of potentially catastrophic problems for hospitals, nursing homes, operating rooms and other critical healthcare facilities.

The only broadbase, broad-spectrum antibiotic known to develop no antimicrobial resistance, during a period of thousands of years of use, is iodine, and, more specifically to the current argument and since 1954 when it was invented, PVPI, also called “povidone iodine” and more technically “polyvinylpyrrolidone iodine.” PVPI is a highly stable compound of polyvinylpyrrolidone and iodine. It has a long multi-year shelf life; it is accepted worldwide as a standard antimicrobial by global institutions such as the World Health Organization, and is present as a central antiseptic preparation in every hospital, physician's office and operating theater in the world. We note that its use has generally been topical, rather than oral.

The present invention proposes to use PVPI, significantly enhanced with our additives and adjuvants, which additives and adjuvants (essential oils, DMSO, non-nutritive sweeteners) constitute a key and indeed a central element of the claims for the present invention.

### BACKGROUND INFORMATION

Antimicrobial resistance (AMR) is considered by the World Health Organization, the Centers for Disease Control and many other national and international governmental and medical institutions to be one of the greatest and most dangerous problems facing the health of mankind. As antibacterial, antic-fungal, anti-yeast and anti-viral drugs have proliferated, so too has antimicrobial resistance.

Many medical and microbiological researchers have commented that AMR threatens to wipe out the antibiotic successes which have defined and dominated modern medicine since the 20^{th} century, as microbes become resistant to our best drugs.

However, this is not the case with iodine and more specifically PVPI. There are no known microbes resistant PVPI.

Many diseases of the recent past—since the 1970's—are recognized as zoonotic, as having an origin in animals other than man. Prior to the 1970's, several prominent medical commentators indicated their belief that the field of infectious diseases was “boring” and all known diseases had been conquered. Shortly thereafter, AIDS/HIV arose, and then avian influenzae. Thus began the great change in the relationship between industrial farming animals, the rapid development of highly infectious and mortal viral diseases, and human uptake of these microbes. The three major currents of globalization, antimicrobial resistance and factory farming have created a fertile ground for the development of an endless array of new, dangerous infectious diseases.

This is especially true of viral diseases such as COVID-19, SARS-CoV, MERS-COV, the avian influenza (AIV), the Ebola virus (EBOV) and others to come. So long as we continue to raise and consume billions of avians and mammals for food, there will be no end to the variety and morbidity of microbial diseases. It should also be noted that the worldwide rise of bioterrorism may further the potential for pandemics.

Highly pathogenic viruses are not readily susceptible to vaccine development, and as we have seen with avian influenzae, the year-to-year variations in viral features make it difficult if not impossible to eradicate these infectious disease organisms. In our view, vaccination is a useful but not the best antimicrobial pathway. Only iodine, as PVPI, remains a bulwark against these dangerous organisms.

### EXAMPLES OF VIRUSES AND OTHER MICROBES TARGETED BY THE PRESENT INVENTION

EBOLA: The Ebola virus, a highly infectious and extremely deadly organism, caused a potential pandemic which killed ˜9,000 in West Africa in 2014, arousing global worry and international relief efforts. This virus belongs to a class of filoviruses, all of which can cause dangerous and deadly infections. The virus is capable of multiplying rapidly without immediate pathological symptoms, and can overwhelm the body's immune system rapidly. It can be spread by aerosolized droplets from one person to another, and can readily infect the respiratory tract. Apparently, only a very few organisms are required for effective infectious transmission, and viable organisms can be isolated from the dead even after a week. The present invention, used diligently, has the potential to significantly improve the safety of healthcare workers and family members when used in combination with protective gear, frequent hand and face-washing, and distancing.

H1N1: Also called “pandemic influenza,” this disease is a potentially fatal infectious condition that continues to raise worldwide concerns. It is apparently a recombinant virus that mixes human virus genes with non-human genes deriving from swine and birds. The industrial farming activities involved in raising large numbers of food animals—close quarters, total and lifelong confinement, breeding for rapid growth, stressful growth conditions, elevated use of antibiotics—have contributed significantly to the develop of highly pathogenic strains of such diseases. Once acquired by a human being, the H1N1 virus, for example, which has been responsible for an estimated 500,000 deaths worldwide in its first year of proliferation, can be readily spread to other humans by the aerosolization of the offending organism by breathing, sneezing, coughing or speaking, or through contact with another infected person's mucous membranes, or with other bodily fluids which may be contaminated with the virus and reside on surfaces.

For the control of these and related viral and bacterial infections, large-scale governmental requirements for facemasks, handwashing, barrier control, and personal protective equipment such as gloves, eye protection, respirators and gowns, have become de rigueur in large group settings (airports, classrooms, churches, etc.) and in healthcare facilities.

In the case of COVID-19, an effective vaccine has been developed, but in order for this vaccine to actually dead-stop the proliferation of the disease, universal and global vaccination is required at a cost of trillions of dollars (USD). There is a definitive and pressing need to protect all manner of personal service providers—from nurses to doctors, from airport service personnel to restaurant waiters—from the potential for disease acquisition and its ensuing costs, complications and human misery.

SARS & MERS: Similar to the avian/swine-derived viruses, Severe Acute Respiratory Syndrome (SARS) and Middle East Respiratory Syndrome (MERS) are both coronaviruses. These are a group of single-stranded RNA enveloped viral organisms with similar but not identical genome sequences. SARS, first recognized in Asia in 2003, was contained from becoming a global outbreak, with nearly 10,000 people affected and about 1,000 expired, usually from pneumonia. Like other viruses which impact the respiratory apparatus, these infectious organisms can be extremely difficult to recognize at first, and often treatment comes too late, and usually is only palliative.

The coronavirus SARS (also called “SARS-CoV”) is transmitted when a person infected with the virus coughs, sneezes, or speaks to others who are nearby, or when their various bodily exudates interact with skin, mouth, nose, eyes or other available and proximate intake areas of the recipient.

MERS, which can cause a mortality rate approaching 40% of the infected population, apparently originated on the Arabian Peninsula (possibly from gerbils, and then spread to camels and to human beings) and, between 2012 and 2015, caused nearly 1300 human cases and 500 deaths. The disease, like other infectious conditions arising from dangerous coronaviruses, can produce symptoms that initiate with minor fever and progress to upper respiratory illness followed by rapid multi-organ failure and death.

As for many of these highly infectious viral diseases, healthcare workers (HCWs) are at extreme risk, as are the families of the infected persons. On a secondary basis, substantial risk applies to emergency medical workers, airport personnel, flight personnel and proximate co-travelers. While recommendations for hand sanitization, gloves, gowns, respirators, eye protection, masks and other personal protective gear may be deployed, often a very small number of these virulent organisms are needed to cause rapid and sometimes fatal infections. The availability of another line of defense such as the present invention has the possibility to become a significant and convenient preventative.

ABOUT ALCOHOL in ANTIMICROBIAL PRODUCTS: Conventional mouthwash and nasal/oral/nasal spray compositions frequently contain ethanol, up to 27% by volume, as a preservative and antimicrobial agent. In some individuals, alcohol may exacerbate xerostomia (dry mouth), which in turn can cause halitosis and significant tooth decay, as the protective effect of saliva's re-mineralizing the enamel is reduced. Xerostomia also makes the mucosa and periodontal tissue of the mouth more vulnerable to infection and increases the risk of gingivitis. Mouthwash and/or nasal/oral/nasal spray comprising high levels of alcohol also may be undesirable for use by alcoholics, children, and members of certain religious faiths, and some consumers may object to the burning sensation of relatively high alcohol compositions. Alcohol, especially ethanol, in a mouthwash represents a significant and perhaps unrecognized danger for non-practicing alcoholics who have given up drinking. As alcoholism is a disease sometimes triggered by alcohol intake, the presence of alcohol in a mouthwash could represent a life-changing danger to non-practicing and unknowing alcoholics. Further, since 12.7% of our U.S. population is alcoholic according to the Centers for Disease Control, the removal of alcohol from a mouthwash is an important “negative feature” of its uniqueness.

A further grave danger in using alcohol in such products is that the target organisms may become resistant to alcohol, and present microbiology indicates that in fact this has happened and will progress. Two microbes—E. faecalis and E. faecium—have become resistant to alcohol, and their resistance is a matter of significant and growing concern to the public health industry. In due course, other organisms will develop alcohol resistance, and it is important to remove alcohol from antimicrobial use whenever possible.

To the best of our knowledge, the present invention is the only product which has eschewed the admixture of any alcohol of any chemical species, for this reason and others provided.

USEFULNESS AND VIABILITY: The vast majority of mouthwash and nasal/oral/nasal spray compositions are neither formulated for nor effective against viruses. Further, the vast majority of mouthwash and nasal/oral/nasal spray compositions are neither formulated for nor effective for a lengthy period of time against microbes; that is, they are not usually effective for a time period greater than approximately 5 minutes.

In contradistinction, PVPI is a well-established broad-spectrum antimicrobial, antiviral, antifungal, anti-mold, anti-virion compound frequently used in the operating theatre in hundreds, if not thousands, of surgical rooms and hospitals daily and in the dental professions. In over 200 years of active use, iodine, and later PVPI after its invention in 1954, has never been shown to produce anti-microbial resistance, that is, the development of microbial serotypes which are resistant to killing by the named substance. Further, PVPI, in contradistinction to alcohol-based antimicrobials, has no record of any resistant organism development; unlike alcohol, which in various forms, including ethanol, isopropanol and other varieties of alcohol, which have recently been shown to develop resistance to common, hospital-ubiquitous and significantly dangerous, life-threatening enterococcal bacteria (E. faecalis and E. faecium). PVPI has been shown to be effective after application for time periods ranging from 3 hours to up to 8 hours, depending upon the area or surface to which it is applied, ambient temperature and humidity, enclosure, bodily areas to which applied and etc. Unlike alcohol and other antimicrobials, enteric iodine is actively absorbed by many bodily organs through the 643-amino acid process called the “sodium iodide symporter” or NIS. The NIS pulls iodine from transient substances in the mouth, digestive system and circulatory systems into interstitial tissue and organs, reserving it for future deployment against infectious disease organisms, including parasites. Iodine is unique in its functionality as both a critically necessary micronutrient and a powerful antiseptic/antibiotic. No other known chemical has this unique activity.

Manufacturers' demonstrations and investigations of polyvinylpyrrolidone iodine (PVPI) have shown that the most effective antimicrobial concentration of PVPI is usually 1.0%, which produces an “available iodine” mixture of molecular and ionic products of 0.1%. In our particular composition, the ratio of molecular to ionic iodine is 1.8:2.0, respectively, a ratio which has been found to be effectively antimicrobial in thousands of reliably measured in vitro and in vivo research and hundreds of recorded anecdotal situations.

In numerous controlled clinical studies, and throughout the research literature in dentistry, maxillofacial surgery, general surgery, pulmonology, cardiology, orthomolecular research, and virtually all fields of medical activity requiring any type of invasive, semi-invasive or irrigation activity, PVPI stands out for its continued and constant power to kill and disintegrate all manner of infectious disease organisms without causing antimicrobial resistance.

Equally important, and totally unique in its activity in the mammalian organism, iodine is a necessary indeed a critically important micronutrient, and is frequently found to be insufficient and deficient in large segments of the world's population. The present invention provides for oral/nasal/salivary gland uptake of iodine to enhance not only the immediate microbial environment, but through storage in the three major sets of human salivary glands, and the 5-10,000 miniscule salivary glands distributed through the mouth, throat and general oral/nasal cavity of human beings, iodine is absorbed and stored for ongoing and continuous use as an immune system enhancer, disabler and killer of all known microbes, including and especially coronaviruses, all envelope and non-enveloped viruses, as well as parasites, fungi and other invasive organisms.

Iodine, at the concentration of the current invention, kills all seven classes of viral organisms. While our particular target interest at this time is the enveloped coronaviruses, and especially COVID-19, all of the classes of viruses below are inactivated by PVPI, at the effective concentration of 0.1% available iodine. At this moment in medical and human history, our country and the world are traversing a horrific pandemic caused by the SARS-CoV-2 virus, or COVID-19. The current invention aims to help to eliminate its transmission and replication at the initial phases of its infection of humans of all ages. We would add, further, that the present invention may also have application to all manner of mammals and ayes, both companion animals (dogs and cats have been known to acquire COVID-19) and industrially-raised food animals (cows, pigs, sheep, goats, chickens, turkeys, etc.). This area of application may in due course represent the largest realm of use and activity of the current invention. The current invention claims a unique position in the area of nasal/oral application to combat the various respiratory diseases, not limited to viral but inclusive of all microbial species: bacteria, viruses, molds, fungi, etc. of both mammals and ayes, which to the best of our knowledge has not been deployed before in the proposed manner.

It is important to note that most rapidly transmissible viral diseases have relatively elevated rates of mortality in human beings and are often, most often, transmitted through respiratorily expelled droplets or mist. Usually such viruses are considered as Level 3 or Level 4 risk groups. Some have the potential to become global pandemics (as in the case of COVID-19), and have a huge impact on society in many ways, and more specifically on healthcare workers (HCWs) and other front-line care providers.

Polyvinyl pyrrolidone iodine in solution has been demonstrated to be effective against all classes of viruses, viz.:

I: dsDNA viruses (e.g. Adenoviruses, Herpesviruses, Poxviruses)
II: ssDNA viruses (+ strand or “sense”) DNA (e.g. Parvoviruses)
III: dsRNA viruses (e.g. Reoviruses)
IV: (+)ssRNA viruses (+ strand or sense) RNA (e.g. Coronaviruses, Picornaviruses, Togaviruses)
V: (−)ssRNA viruses (− strand or antisense) RNA (e.g. Orthomyxoviruses, Rhabdoviruses)
VI: ssRNA-RT viruses (+ strand or sense) RNA with DNA intermediate in life-cycle (e.g. Retroviruses)
VII: dsDNA-RT viruses DNA with RNA intermediate in life-cycle (e.g. Hepadnaviruses)

### BRIEF SUMMARY OF THE INVENTION

The invention provides an ethanol-free liquid oral/nasal care composition comprising polyvinyl pyrrolidone iodine, mixed with stevia sweetener and peppermint oil and dimethyl sulfoxide (DMSO). This pleasant-tasting invention's taste acceptance is accomplished by admixing relatively insoluble flavoring oils (e.g., peppermint oil) with the PVPI in a particular ratio along with DMSO, stevia sweetener and water.

For example, the invention provides a liquid oral/nasal/nasal care composition which is substantially free of ethanol, comprising
(i) An antimicrobially effective amount and dilution of PVPI; and
(ii) One or more flavoring oils/essential oils which are generally insoluble in water at room temperature, wherein the ratio of the PVPI I to the one or more flavoring oils is variable. Without intending to be bound by theory, it is believed, that at optimal concentrations, the PVPI and the one or more essential oils and the stevia sweetener spontaneously or with gentle mixing can (and indeed apparently do) form a stable micelle or a water-in-oil microemulsion at room temperature in the presence of water, which helps mask the bitter taste of the PVPI without affecting its efficacy as an antibacterial agent, and at the same time, the surfactant properties of the PVPI allow for elevated effective levels of poorly soluble oils which themselves may have antimicrobial properties. It is noted that many essential oils, and particularly peppermint, have significant vasodilation action. The result of their use is that the penetration activity of the PVPI is enhanced by their presence, as it is by the dimethyl sulfoxide (DMSO);
(iii) DMSO—dimethyl sulfoxide—a powerful solvent, and penetrating liquid, that serves three key functions: a) to effectively dissolve and allow the homogeneous distribution of the essential oil or oils; b) to effectively dissolve and allow the homogeneous distribution of the Stevia (see (v) below); and c) to penetrate the mucosal tissues of the receiving person and rapidly carry the active antimicrobial products—PVPI and peppermint (or other essential oils)—directly to affected or potentially affected tissues;
(iv) Water (generally pure ion-free distilled USP water); and
(v) Stevia sweetener, initially mixed as powder and dissolved in the remaining liquid elements of the invention after first being dissolved in the DMSO.

Any description and specific examples, while indicating the preferred embodiment of the invention, are intended for purposes of illustration only and are not intended to limit the scope of the invention.

Note that the liquid oral/nasal/nasal care composition described above will also be used in other frameworks to provide similarly effective antimicrobial action.

For example, the present invention incorporates the use of a certain number of drops of the liquid oral/nasal/nasal care composition in lozenges and other oral vehicles (candies, suckers, lollipops, wafers, etc. without reservation) to deliver a continuous, measured flow of antimicrobial product to the mouth and throat. The lozenge element of the invention will be initially expressed in three versions, based on size and antimicrobial strength: small, medium, and large. The second iteration (medium) will deliver 2× the antimicrobial product of the first (small) iteration; the third iteration will deliver 2× the antimicrobial product of the second (medium) iteration.

The various concentrations of the antimicrobial product in the lozenge (or other candy-type solid carrier) may require that the product itself is color-coded, and that the packaging and boxing reflect the three various concentrations of the product.

It is also noted that this vehicle for easy and acceptable delivery of the liquid oral/nasal/nasal care composition serves another related and extremely important process: the process of helping to prevent iodine deficiency, which affects over 2 billion people on earth, according to the World Health Organization. Additionally, over 15% of all women in the United States of child-bearing age are likewise iodine deficient, according to the U.S. Centers for Disease Control, and throughout the course of their pregnancy they and their unborn and nursing babies could benefit from the use of the lozenge form of the present invention.

### DETAILED DESCRIPTION OF THE INVENTION

In one preferred embodiment, Composition 1, the invention provides a liquid oral/nasal care composition which is totally free of ethanol, comprising (i) an antimicrobially effective amount of polyvinylpyrrolidone iodine (PVPI); (ii) DMSO as a diluent, solvent, carrier and penetrant; and (ii) one or more flavoring oils which are substantially insoluble, e.g., less than 1% soluble, in water at room temperature but effectively soluble and capable of homogeneity in DMSO, wherein the ratio of the PVPI to the one or more flavoring oils is from 1:0010 to 1:0020 or such concentration as to provide a pleasant but not an overwhelming taste of essential flavoring oil, and for which the DMSO will act as a carrier/solvent and (iv) Stevia sweetener, and (v) water.

In general, the invention deploys standard hospital-quality pre-surgical PVPI antiseptic wash as used in the hospital operating theatre and throughout the medical community, and typically usually provided to the medical field in a 10% concentration. This concentration of PVPI product typically contains four or five stabilizing compounds which help to extend the homogeneity and the shelf life of the PVPI provided. The present invention does not contemplate changing these elements, but in using the PVPI “as it comes” from the provider along with its stabilizers and homogenizers. None of these have any negative or positive antimicrobial activity as determined by the manufacturing providers.

The present invention dilutes this 10% concentration to between 1.0%-3.5%. While it is microbiologically recognized, and well-documented by the PVPI manufacturers, that a 1% concentration of PVPI provides the highest and most rapid antimicrobial action, the present invention may use a higher concentration because within the confines of the nose, mouth and throat the PVPI will always become substantially diluted by saliva, mucous and other bodily fluids, and while it is impossible to estimate the actual dilution that will occur, given the variation of liquid in human mucosal tissues, the small amounts of PVPI present in the product may well be diluted by up to 50%. There is no way to know exactly what this will be, as the elements of individual human variation are infinite.

The present invention contemplates providing various concentrations of PVPI in its products. The consumer or physician's choice as to which concentration is chosen to be used will depend upon the medical condition and mucus/saliva production, among other factors, and the experience of the user or physician.

For example, the invention provides
1.1. The liquid, oral/nasal care composition of Composition 1 wherein the ratio of the PVPI to the one or more essential flavoring oils is 1:0.0010 to 1:0.0020 or such concentration as would be necessary to provide a pleasant but not an overwhelming taste of essential flavoring oil.
1.2. The liquid oral/nasal care composition of Composition 1 wherein the PVPI and the one or more essential flavoring oils form a micelle microemulsion within the DMSO at room temperature prior to being added to water and PVPI.
1.3. The liquid oral/nasal care composition of the foregoing wherein the one or more essential flavoring oils, which serve both vasodilation and flavoring features of the invention, comprise peppermint oil.
1.4. The liquid, oral/nasal care composition of the foregoing Composition wherein the one or more flavoring oils comprise menthol, spearmint oil, peppermint oil, or combinations of any of these.
1.5. The liquid oral/nasal care composition of the foregoing Composition wherein the one of more flavoring oils are selected from one or more of menthol, spearmint oil, peppermint oil, carvone, methyl salicylate, anethole, thymol, eugenol, coolants, cinnamic aldehydes, and mixtures thereof.
1.6. The liquid oral/nasal care composition of the foregoing Composition wherein the one or more flavoring oils comprise a combination of menthol, spearmint oil and peppermint oil in a ratio to PVPI that may differ by 20-50% in either direction.
1.7. The liquid oral/nasal care composition of any of the foregoing Compositions that is free of ethanol and that comprises
   an antimicrobially effective amount of polyvinylpyrrolidone iodine in the range of 1.0% to 3.5% of the total volume; and
   an effective amount of an essential oil flavoring component the flavoring component comprising
      menthol, e.g. in an amount of at least 15%, 15-25%, e.g. about 20% by weight of essential oil flavoring component;
      peppermint oil (e.g., natural peppermint oil, synthetic peppermint oil, cornmint oil or mixtures thereof), e.g., in an amount of at least 10%, e.g., 10-20%, e.g., about 15% by weight of the essential oil flavoring component;
      spearmint oil, e.g., in an amount of 0-55%, e.g., 40-50%, about 45% by weight of flavoring component;
1.8. The liquid oral/nasal care composition of any of the foregoing Compositions (e.g. mouthwashes, toothpastes, throat sprays, and breath sprays) being free of ethanol and comprising an antimicrobially effective amount of a PVPI and essential oil vasodilators/flavorants in the following ratio:
   1 part PVPI:
   0.3-0.9, e.g., 0.5-0.7 parts menthol:
   0.4-1.2, e.g., 0.5-0.8 parts spearmint oil:
   0.1-0.5, e.g., 0.2-0.4 parts peppermint oil;
   and optionally further ingredients, e.g. further comprising one or more of additional flavorants, e.g., comprising one or more of anethole (e.g., trans-anethole), thymol, methyl salicylate, coolants (for example n-ethyl-p-methane-3-carboxamide, Optacool®), cinnamic aldehydes and mixtures thereof;
   non-caloric sweeteners, e.g., comprising Stevia, sugar alcohols (e.g., sorbitol), or mixtures thereof;
water.
1.9. The liquid oral/nasal care composition of any of the foregoing Compositions in the form of a mouthwash and nasal/oral spray;
1.10. The liquid oral/nasal care composition of any of the foregoing Compositions comprising PVPI and an essential oil flavor component comprising flavorants in a ratio of PVPI and flavorants by weight is about 1 part PVPI about 0.3 parts menthol:about 0.7 parts spearmint oil:about 0.24 parts peppermint oil; wherein the composition may optionally comprise coolants and/or sweeteners in addition to the flavor component.
1.11 The liquid oral/nasal care composition of any of the foregoing Compositions wherein the flavor component comprises by weight of essential oil flavor component about 20% menthol, about 45% spearmint oil and about 15% peppermint oil;
1.12. The liquid oral/nasal care composition of any of the foregoing Compositions comprising L-menthol;
1.13. The liquid oral/nasal care composition of any of the foregoing Compositions comprising menthol in addition to the menthol in the essential oil flavoring component, e.g., 0.01-0.04, e.g., about 0.02% menthol by weight of the total composition, in addition to the menthol in the essential oil flavoring component;
1.14. A composition according to any of the preceding Compositions comprising 0.05-0.07% total menthol, e.g., including added menthol and menthol from peppermint oil and spearmint oil;
1.15. A composition according to any of the preceding embodiments wherein the flavoring component further comprises an anethole, e.g., trans-anethole, in an amount of 1-10%, e.g., 2-6%, e.g., about 4% by weight of flavoring component; or 0.001-0.01%, e.g., 0.002-0.006% e.g., about 0.004% by weight of the total composition;
1.16. A composition according to any of the preceding embodiments which is an aqueous solution, water-in-oil emulsion, water-in-oil microemulsion, or micellar system including its admixture with DMSO;
1.17. A composition according to any of the preceding embodiments which is a water-in-oil microemulsion;
1.18. A composition according to the preceding embodiments which is a micellar system;
1.19. A composition according to any of the preceding embodiments further comprising additional flavorants, e.g., comprising one or more of anethole (e.g., trans-anethole), thymol, coolants (for example comprising menthol, n-ethyl-p-methane-3-carboxamide, and combinations thereof), cinnamic aldehydes and mixtures thereof, for example coolant mixtures available commercially, e.g. Optacool® (Symrise);
1.20. A composition according to any of the preceding embodiments further comprising non-caloric sweeteners, e.g., comprising Stevia, sugar alcohols (e.g., sorbitol), or mixtures thereof.
1.21. A composition according to any of the preceding embodiments further comprising humectants, e.g., comprising propylene glycol, glycerin or mixtures thereof;
1.22. A composition according to any of the preceding embodiments comprising water, colorant, Stevia, PVPI, poloxamer, e.g., Pluronic. F-127, sorbitol, glycerin, propylene glycol, parabens, and menthol, wherein the composition is light brown or tan and substantially clear.
1.23. A composition according to any of the preceding embodiments further comprising non-ionic surfactants, e.g., poloxamers, e.g., poloxamer 470;
1.24. A composition according to any of the preceding embodiments which is an aqueous solution, water-in-oil emulsion, or water-in-oil microemulsion including DMSO.

The invention further provides methods of prophylaxis and/or treatment of a disease or condition selected from one or more of infection by enveloped, non-enveloped and other viral entities, dry mouth, halitosis, gingivitis, tooth decay, and cavities, comprising administering an effective amount of a composition as hereinbefore described, e.g. any of Compositions 1, 1.1, et seq., to a subject in need thereof.

The invention further provides the use of polyvinylpyrrolidone iodine (PVPI) and one or more flavoring oils which are substantially insoluble in water at room temperature, however, in this invention they are effectively dissolved in DMSO and used in the manufacture of a liquid oral/nasal care composition, e.g. any of Compositions 1, 1.1 et seq., for prophylaxis and/or treatment of a disease or condition selected from one or more of infection by enveloped, non-enveloped and other viral entities, dry mouth, halitosis, gingivitis, tooth decay, and cavities.

The invention further provides a method of making a composition e.g. any of Compositions 1, 1.1, et seq., comprising adding flavoring oils, dissolved in a small amount of DMSO, to an aqueous solution comprising polyvinylpyrrolidone iodine and mixing until the composition is clear, e.g., a method comprising adding DMSO to water and mixing, then adding Stevia, and PVPI and mixing.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

In another preferred embodiment, as shown in the drawing of FIG. 1, a human medical face mask 10 is shown with a pair of ear encircling loops 12 and 14 on each end thereof. The mask 10 has a face facing portion 16 when the mask 10 is being worn by a person. At least one perforated or foraminous pocket 18 is arranged on the face facing side of thee mask 10. The pocket is arranged to hold an ampule or capsule 20 containing the PVPI product. The ampule or capsule 20 is squeezable or crushable for a mask wearer or user to squeeze and release a spray of PVPI product towards onto or into the mask wearer's mask 10 or his/her nasal/oral area for enabling masked distribution of an antimicrobial product of the present invention.

Unless otherwise specified, all percentages and amounts expressed herein and elsewhere in the specification should be understood to refer to percentages by weight. The amounts given are based on the active weight of the material.

### EXAMPLE 1

### Comparative Compositions

Compositions of varying strengths are prepared with different essential flavoring oils as follows:

Ex A: Mouthrinse composition Ingredients: PVPI, Stevia, Essential Flavoring Oil,

DMSO, Water.

Ex B: Flavoring component Ingredients: L-menthol, North American peppermint oil, North American spearmint oil, Synthetic peppermint oil, Cornmint oil, N-ethyl-p-methane, carboxamide, Trans-anethole, Eugenol, Thymol, Cinnamic aldehydes, Methyl salicylate

### EXAMPLE 2

### Consumer Testing

The Compositions of the previous examples (in various formulations) were tested with consumers to measure flavor liking and purchase intent. It was found that the more acceptable Compositions had higher levels of menthol and peppermint oil relative to spearmint oil. Spearmint oil and peppermint oil differ, inter alia, in that peppermint oil has higher amounts of menthol.

Without being bound by theory it is hypothesized that the relatively higher levels of poorly soluble menthol permit formation of a spontaneous microemulsion or micellar system incorporating the PVPI, reducing the perception of bitter taste from the PVPI. At the same time, the PVPI allows the composition to contain higher levels of poorly soluble oils such as menthol, which themselves have antibacterial properties, thereby enhancing the efficacy of the composition. The invention contemplates to use always DMSO as a solvent/carrier for the relatively immiscible oils and to assist in the homogenization of the final product. Perfect homogenization does not occur.

The invention thus comprises an ethanol-free virus treatment composition, comprising: an antimicrobially effective amount of polyvinylpyrrolidone iodine (PVPI) ranging from 1.0% to a dilution-accommodating 3.5%; inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes; a Stevia sweetener pre-mixed in a microemulsion solution of dimethyl sulfoxide (DMSO); and ion-free distilled water. The ratio of PVPI and DMSO solution to the inclusion of essential flavoring oil is 1:0010 to 1:0020. The ethanol-free virus treatment composition may be a nasal spray. The ethanol-free virus treatment composition may be a mouthwash. The ethanol-free virus treatment composition may be formed into a lozenge. The lozenge may comprise multiple layered concentrations of the composition. The microemulsion solution of DMSO is preferably no greater than 1% by weight. The ethanol-free virus treatment composition may be contained in a compressible composition spray-dispersible capsule. The compressible composition spray-dispersible capsule may be supportively arranged on a face facing side of a medical face mask.

The invention also comprises a method of making an ethanol-free Covid-19 oral/nasal treatment composition, comprising the steps of: mixing an antimicrobially effective amount of polyvinylpyrrolidone iodine (PVPI) ranging from 1.0% to a dilution accommodating 3.5% with an inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes; with a Stevia sweetener pre-mixed in a microemulsion solution of dimethyl sulfoxide (DMSO); and a measure of ion-free distilled water until the color of the treatment composition is a light tan. The method of making an ethanol-free Covid-19 oral/nasal treatment composition, may include the step of: mixing an antimicrobially effective amount of polyvinylpyrrolidone iodine (PVPI) ranging from 1.0% to a dilution accommodating 3.5% with an inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes; with a Stevia sweetener pre-mixed in a microemulsion solution of dimethyl sulfoxide (DMSO); and a measure of ion-free distilled water until the color of the treatment composition is a light tan, for inclusion into a lozenge. The method of making an ethanol-free Covid-19 oral/nasal treatment compos may include the step of: mixing an antimicrobially effective amount of polyvinylpyrrolidone iodine PVPI ranging from 1.0% to a dilution accommodating 3.5% with an inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes; with a Stevia sweetener pre-mixed in a microemulsion solution of dimethyl sulfoxide (DMSO); and a measure of ion-free distilled water until the color of the treatment composition is a light tan, and into a face mask-attachable compressible composition spray-dispersible capsule.

The invention may also comprise an ethanol-free Covid-19 oral/nasal treatment composition, consisting of: an antimicrobially effective amount of polyvinylpyrrolidone iodine (PVPI) ranging from 1.0% to a dilution-accommodating 3.5%; an inclusion of at least one room-temperature, water-insoluble, viscous membrane permeation-enhancing essential flavoring oil selected from the group consisting of: menthol, spearmint oil, peppermint oil, carvone, methyl salicylate, anethole, thymol, eugenol, coolants and cinnamic aldehydes; a Stevia sweetener pre-mixed in a microemulsion solution of dimethyl sulfoxide (DMSO); and ion-free distilled water. The menthol is in the composition preferably is in an amount of at least 15% by weight, wherein the spearmint oil is in the composition in an amount of between 30% to 55% by weight, and wherein peppermint oil is in the composition in an amount of at least 10% by weight.

The invention also may comprise an ethanol-free antimicrobial hand-sanitizer treatment composition, comprising: an antimicrobially effective amount of polyvinylpyrrolidone iodine (PVPI) ranging from 1.0% to a dilution-accommodating 3.5%; an inclusion of a microemulsion solution of dimethyl sulfoxide (DMSO); an inclusion of Guar gum in a ratio of between 1:4 and 1:6 times the volume of DMSO; and ion-free distilled water wherein the distilled water comprises a range of 6 to 12 times the volume of DMSO.

The invention also comprises an ethanol-free antimicrobial eye treatment composition, for eye dropper to eye application, the composition comprising: an antimicrobially effective amount of polyvinylpyrrolidone iodine (PVPI) ranging from about 1.0% to about 2.5% by volume of the composition; an inclusion of a microemulsion solution of dimethyl sulfoxide (DMSO) ranging between 1% to 44% of the total volume of the composition; and an inclusion of a volume of ion-free distilled water to bring the total volume of the inclusive PVPI and DMSO composition to 100%.

The invention also comprises a human-face-attachable virus-inhibiting face mask having a first or outer side and a second or face facing side, the second side having a compressible virus-destroying composition-containing spray-facilitating capsule-supporting foraminous pocket thereon, so as to enable a mask wearer to manually compress the capsule contained within the foraminous pocket to spray release the virus-destroying composition containing capsule against a nasal or oral facial area of the mask wearer. The human-face-attachable virus-inhibiting face mask virus-destroying composition preferably comprises an ethanol-free virus treatment composition, comprising: an antimicrobially effective amount of polyvinylpyrrolidone iodine (PVPI) ranging from 1.0% to a dilution-accommodating 3.5%; an inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes; a Stevia sweetener pre-mixed in a microemulsion solution of dimethyl sulfoxide (DMSO); and ion-free distilled water.

1. An ethanol-free virus treatment composition, comprising:
an antimicrobially effective amount of polyvinylpyrrolidone iodine (PVPI) ranging from 1.0% to a dilution-accommodating 3.5%;
inclusion of at least one room-temperature, water-insoluble, viscous membrane permeation-enhancing essential flavoring oil selected from the group consisting of: menthol in an amount of at least 15% by weight, spearmint oil in an amount of between 30% to 55% by weight, peppermint oil in an amount of at least 10% by weight, carvone, methyl salicylate, anethole, thymol, eugenol, coolants and cinnamic aldehydes;
a Stevia sweetener pre-mixed in a microemulsion solution of dimethyl sulfoxide (DMSO); and
ion-free distilled water.

2. The ethanol-free virus treatment composition as recited in claim 1, wherein the ratio of PVPI and DMSO solution to the inclusion of essential flavoring oil is 1:0010 to 1:0020.

3. The ethanol-free virus treatment composition as recited in claim 1, is a nasal spray.

4. The ethanol-free virus treatment composition as recited in claim 1, is a mouthwash.

5. The ethanol-free virus treatment composition as recited in claim 1, is formed into a lozenge.

6. The ethanol-free virus treatment composition as recited in claim 5, wherein the lozenge is comprised of multiple layered concentrations of the composition.

7. The ethanol-free virus treatment composition as recited in claim 1, wherein the microemulsion solution of DMSO is no greater than 1% by weight.

8. The ethanol-free virus treatment composition as recited in claim 1, wherein the treatment composition is contained in a compressible composition spray-dispersible capsule.

9. The ethanol-free virus treatment composition as recited in claim 8, wherein the compressible composition spray-dispersible capsule is supportively arranged on a face facing side of a medical face mask.

10.-12. (canceled)

13. An ethanol-free Covid-19 oral/nasal composition for the treatment of mammals in general, and farm animals in particular, consisting of:
an antimicrobially effective amount of polyvinylpyrrolidone iodine (PVPI) ranging from 1.0% to a dilution-accommodating 3.5%;
inclusion of at least one room-temperature, water-insoluble, viscous membrane permeation-enhancing essential flavoring oil selected from the group consisting of: menthol, spearmint oil, peppermint oil, carvone, methyl salicylate, anethole, thymol, eugenol, coolants and cinnamic aldehydes;
a Stevia sweetener pre-mixed in a microemulsion solution of dimethyl sulfoxide (DMSO); and
ion-free distilled water.

14. The ethanol-free Covid-19 oral/nasal treatment composition, as recited in claim 13, wherein the menthol is in the composition in an amount of at least 15% by weight, wherein the spearmint oil is in the composition in an amount of between 30% to 55% by weight, and wherein peppermint oil is in the composition in an amount of at least 10% by weight.

15.-19. (canceled)
